(19) United States
(12) Patent Application Publication
(10) Pub. No.: US 2003/0138435 A1
(43) Pub. Date: Jul. 24, 2003

(54) Trisomy 21 cytotrophoblast cultures,and uses thereof for obtaining trisomy 21 markers

(76) Inventors: Daniele Evain-Brion, Bourg-La-Reine (FR); Jean-Louis Frendo, Paris (FR)

Correspondence Address: MORGAN LEWIS & BOCKIUS LLP, 1111 PENNSYLVANIA AVENUE NW, WASHINGTON, DC, 20004, US

(21) Appl. No.: 10/220,433

(22) PCT Filed: Mar. 2, 2001
(86) PCT No.: PCT/FR01/00619

(30) Foreign Application Priority Data
Mar. 3, 2000 (FR) 00/02795

(51) Int. Cl. A61K 39/00; A61K 39/38

(52) U.S. Cl. 424/184.1

(57) ABSTRACT

The invention concerns trisomy 21 cytotrophoblast cultures. Proteins differentially secreted by said cultures and by normal cytotrophoblast cultures are in particular useful as trisomy 21 markers.

The invention relates to trisomy 21 cytotrophoblasts cultures, and to uses thereof.

Trisomy 21, responsible for Down's syndrome which is more commonly called mongolism, is the most frequent of autosomal trisomies since observed in nearly 1 out of 800 neonates during childbirth [FERGUSON-SMITH et al., Prenat. Diagn., 4, 5-44, (1984)]. It is the major genetic cause of mental retardation [CUNNINGHAM et al., Williams Obstetrics, ed. 19, 919-938, (1993)].

Prenatal diagnosis of trisomy 21 is essentially based on the establishment of the fetal karyotype, an examination most often performed on fetal cells of the amniotic fluid collected by amniocentesis, more rarely on trophoblast cells collected by biopsy of the chorionic villi.

However, the establishment of the fetal karyotype cannot be systematically performed, and has for a long time been reserved for women who are 38 years old and over and/or those exhibiting warning signs from ultrasound scans. Some warning signs from ultrasound scans, and in particular the thickening of the nape of the neck of the fetus during the first trimester of pregnancy indeed make it possible to detect 80% of cases of trisomy 21 [NICOLAIDES et al., B.M.J., 304, 867-869, (1992); NICOLAIDES et al., Br. J. Obstet. Gynecol., 101, 782-786, (1994); SNIJDERS et al., Lancet, 352, 343-346, (1998)]. However, the efficacy of this mode of screening depends directly on the expertise of the operator. Moreover, although the risk of trisomy 21 increases with age, nearly 70% of trisomy 21 children are born to mothers under 35 years of age [WALD et al., Brit. Med. J., 297, 883-887, (1988)].

Under these conditions, it has been proposed to supplement these examinations by assaying maternal serum markers, with the aim in particular of carrying out prior detection of cases in which the establishment of the fetal karyotype would be recommended.

In 1987, BOGART et al. [Prenat. Diagn., 7, 623-630] described, in two thirds of pregnancies with a trisomic fetus, a reduction in α-fetoprotein associated with a rise in hCG in the maternal serum in the second trimester. Numerous studies have since confirmed the link between the risk of trisomy 21 and high hCG, and in particular βhCG, levels in the mother [BROCK et al., Prenat. Diagn., 10, 245-251, (1990); CHARD et al., The embryo. Springer Verlag, London, 209-226, (1991); HADDOW et al., N. Engl. J. Med., 338, 955-961, (1998)]. Moreover, low levels of nonconjugated estriol also exist in the maternal blood [HADDOW et al., N. Engl. J. Med., 327, 588-593, (1992); HADDOW et al., N. Engl. J. Med., 330, 1114-1118, (1994); VAN-LITH, Prenat. Diagn., 12, 495-504, (1992)]. These three markers not being interdependents, it is possible to calculate the risk of a fetal trisomy 21 by combining their determination; this method of screening is called “triple test”.

It should be noted however that the use of nonconjugated estriol as an additional marker in the “triple test” is much debated [cf. for example SPENCER, Screening for Down's syndrome, Cambridge University Press, 141-162, (1994)] and, for reasons of public health savings, its determination is not systematically carried out.

These tests make it possible to screen slightly more than 60% of trisomy 21 cases; about 5% of false-positives are in addition observed whose fetal karyotype will in fact prove normal.

It therefore appears desirable to have other trisomy 21 serum markers which would make it possible to improve the sensitivity and/or the specificity of the screening.

Moreover, the origin of the abnormal maternal serum hormonal profile observed in trisomy 21 remains completely unknown.

For example, it was assumed that the increase in the circulating hCG levels in the mother in the case of trisomy 21 could result from an increased production by the trophoblast at a given gestational age, or from a reduction in the clearance of the molecule related to a post-translational modification. Thus, ELDAR-GEVA et al. [J. Clin. Endocrinol. Metab., 80, 3528-3531, (1996)] report a very high increase in the expression and secretion of hCG in trisomy 21 trophoblast cells collected in the second trimester of pregnancy, and propose it as one of the possible causes of the increase in maternal hCG. Other authors [BRIZOT et al., Mol. Hum. Reprod., 10, 2506-2509, (1995)] do not observe a difference in the expression of hCG between normal placentas and trisomy 21 placentas in the first trimester of gestation. COLE et al. [Prenatal. Diagnosis, 18, 926-933, (1998)] have demonstrated the presence, in maternal urines of trisomy 21 cases, of a hyperglycosylated form of hCG similar to that produced in the case of choriocarcinoma; these authors propose the serum or urine assay of this hyperglycosylated form for the detection of trisomy 21.

It therefore appears that few studies have been carried out on the molecular abnormalities observed in the trisomy 21 placenta, and that the results thereof are contradictory.

The aim of the present invention is to provide novel means of studying the cellular and molecular dysfunctions occurring during placental development in trisomy 21, as well as novel trisomy 21 markers. For this purpose, the inventors cultured cytotrophoblast cells derived from normal placentas and from placentas with fetal trisomy 21.

The cytotrophoblast cell constitutes the key cell of human placenta. It differentiates in vitro and in situ in the placenta into syncytiotrophoblast. The syncytiotrophoblast is the endocrine tissue of human placenta. It produces steroid and polypeptide hormones in very high levels (several grams per day).

In vitro, cytotrophoblasts isolated and purified from normal placenta aggregate and then fuse to form the syncytiotrophoblast. This cell, which is highly polarized, secretes in vitro in the culture medium the hormones which it normally secretes into the maternal circulation.

The inventors have isolated, purified and cultured cytotrophoblasts of trisomy 21 placentas at all the terms of pregnancy, and compared their development and their capacity to express and secrete various peptide hormones, with that of normal cytotrophoblasts. They have thus observed that cytotrophoblast cultures derived from trisomy 21 placentas did not form syncytiotrophoblast, or formed a defective syncytiotrophoblast, and that this formation occurred much later than in cytotrophoblast cultures derived from normal placentas removed at the same stage of gestation. They furthermore measured the expression and secretion, in the culture medium, of some (αhCG, βhCG, hPL, PGH and leptin) of the peptide hormones normally produced by the trophoblast, and observed that in the cytotrophoblast cultures derived from trisomy 21 placentas, the abnormalities in the formation of syncytiotrophoblast were accompanied by a substantial decrease in these hormones. These results being in contradiction with those previously reported by ELDAR-GEVA et al. (abovementioned publication), the inventors, in order to verify the absence of artifact, measured the expression of the peptide hormones mentioned above in samples of placental tissues. They thus observed in the tissues of trisomy 21 placentas a level of expression far less than the average level of expression of the same hormones in the tissues derived from normal placentas.

In addition, they observed that transformed cytotrophoblasts, overexpressing copper and zinc dependent superoxide dismutase (Cu/Zn SOD), exhibited the same abnormalities in the formation of the syncytiotrophoblast, and a decrease in the production of αhCG, βhCG, hPL and PGH. In humans, the gene for Cu/Zn SOD is carried by the long arm of chromosome 21, and this enzyme forms part of those which are over-expressed in subjects affected by trisomy 21.

The subject of the present invention is a cytotrophoblast culture, characterized in that said cytotrophoblasts are trisomic for human chromosome 21, or for a region thereof comprising at least the gene encoding Cu/Zn superoxide dismutase, and in that they express, after 3 days of culture, at least one peptide hormone chosen from human placental lactogen (hPL), α human chorionic gonadotrophin (αhCG), β human chorionic gonadotrophin (βhCG), human placental growth hormone (PGH), and leptin, in a quantity less than that expressed on average by normal cytotrophoblasts derived from placentas at the same stage of gestation, and cultured under the same conditions.

The expression “normal cytotrophoblasts” is understood here to mean cytotrophoblasts exhibiting a normal karyotype, and which are capable of forming a syncytiotrophoblast when they are cultured in vitro.

Cytotrophoblast cultures in accordance with the invention may be obtained from trisomy 21 placentas by an enzymatic digestion in several stages, followed by purification on a Ficoll gradient.

The cells thus isolated may then be conventionally cultured, according to the techniques customarily used for normal cytotrophoblast cultures.

Cytotrophoblast cultures in accordance with the invention can be used as a model in vitro for studying abnormalities in the differentiation of the syncytiotrophoblast in trisomy 21, for example the abnormalities of cell fusion. In particular, as the trophoblast cells secrete into the culture medium the same soluble proteins as those which they secrete in vivo into the maternal blood or into the fetal compartment, the cytotrophoblast cultures in accordance with the invention can be used to identify and characterize biological molecules, in particular proteins, released into the maternal blood by placentas of trisomy 21 fetuses, and which are quantitatively and/or qualitatively different from the biological molecules released into the maternal blood by placentas of normal fetuses, and which can thus constitute trisomy 21 markers.

It is possible to analyze in particular the differential expression of the genes and of the proteins, between cultures of normal trophoblast cells and cultures of cells in accordance with the invention, during various stages of formation of the syncytiotrophoblast, including the development of the differentiated syncytiotrophoblast.

For example, cytotrophoblast cultures in accordance with the invention may be used for obtaining libraries for differential expression of nucleic acids between normal cytotrophoblasts and cytotrophoblasts which are trisomic for chromosome 21, and/or between cytotrophoblasts which are trisomic for chromosome 21 at different stages of culture.

The present invention thus includes libraries for differential expression of nucleic acids obtained with the aid of at least one culture of cytotrophoblasts in accordance with the invention. Such libraries may consist for example:

of a population of total or partial cDNA sequences of genes which are expressed in the cytotrophoblast cultures in accordance with the invention, and which are not expressed in normal cytotrophoblast cultures at the same stage of formation of the syncytiotrophoblast;

of a population of total or partial cDNA sequences of genes which are not expressed in the cytotrophoblast cultures in accordance with the invention, and which are expressed in normal cytotrophoblast cultures at the same stage of formation of the syncytiotrophoblast;

of a population of total or partial cDNA sequences of genes whose expression is greater in the cytotrophoblast cultures in accordance with the invention than in normal cytotrophoblast cultures at the same stage of formation of the syncytiotrophoblast;

of a population of total or partial cDNA sequences of genes whose expression is lower in the cytotrophoblast cultures in accordance with the invention than in the normal cytotrophoblast cultures at the same stage of formation of the syncytiotrophoblast;

of a population of total or partial cDNA sequences of genes whose expression varies between different stages of formation of the syncytiotrophoblast in cell cultures in accordance with the invention;

of a population of total or partial cDNA sequences of genes whose expression in cell cultures in accordance with the invention varies according to the environmental conditions, or in response to the application of a physical or chemical agent (for example at 2 different temperatures, in the presence or in the absence of oxygen, and the like).

Libraries for differential expression of nucleic acids in accordance with the invention may be obtained by methods known per se to a person skilled in the art. By way of nonlimiting examples, there may be mentioned, for example, methods such as DDRT-PCR [LIANG and PARDEE, Science, 257, 967-971, (1992)], subtractive hybridization [JIANG and FISHER, Mol. Cell. Different., 1, 285-299, (1993)], RAP-PCR [MCCLELLAND and WELSH, PCR Methods & Applications, 4, S66-81, (1994)], SAGE analysis [VELCULESCU et al., Science, 270, 484-487, 1995], and different variants thereof. They may then be used in particular to identify, characterize and clone genes involved in syncytiotrophoblast differentiation abnormalities, and/or to study the mechanisms of regulation of these genes.

It is also possible, in order to study the genes involved in the differentiation of the syncytiotrophoblast, and the transcription factors regulating these genes, to use nuclear extracts prepared from cultures of cytotrophoblasts in accordance with the invention or of syncytiotrophoblasts formed by these cultures.

Cytotrophoblast cultures in accordance with the invention may also be used to study the differential expression and/or secretion of proteins during various stages of formation of the syncytiotrophoblast, by comparing their protein expression and/or secretion profiles with those of normal cytotrophoblast cultures, or with those of other cytotrophoblast cultures in accordance with the invention, at another stage of development or cultured under different conditions.

It is possible, for example, to compare the quantity of proteins produced and/or secreted, as well as the qualitative differences between proteins, in particular post-translational modifications. The comparison may be carried out on total proteins, or on subpopulations of proteins, for example on the intracellular proteins or on those secreted into the culture medium, on the proteins glycosylated or on those which are not, and the like.

The methods of separation, detection and quantification or qualitative analysis which make it possible to carry out this comparison are known per se to persons skilled in the art.

There may be mentioned, for example, two-dimensional electrophoresis, optionally coupled to mass spectrometry, which makes it possible to rapidly identify, by comparing the electrophoresis gels, the differences between two protein expression profiles. Likewise, the glycosylation or the absence of glycosylation of a protein may for example be detected by isoelectric focusing; according to their glycosylation (in particular their sialic acid content), the different glycoforms will migrate until they arrive in a pH zone corresponding to their isoelectric point; this isoelectric focusing profile may be compared with that obtained after treatment with an enzyme (for example neuraminidase or mannosidase) which hydrolysis the glycans. It is also possible to detect the glycosylated proteins by affinity electrophoresis on a gel containing a lectin; the glycoforms are separated according to their affinity for the lectin.

The proteins separated on gel may, for example, be detected by conventional staining or by immunodetection after transfer onto nitrocellulose, and their quantity may be evaluated by densitometry. The proteins for which a differential expression is observed may then be purified from gels in order to complete their identification and their characterization.

It may also be possible to analyze the proteins of the culture medium by chromatography. For example, to detect and/or quantify glycosylated proteins, it is possible to carry out affinity chromatography on lectin.

By way of example, comparison of the expression of the mRNAs, and of the secretion of the proteins in cytotrophoblast cultures in accordance with the invention and in normal cytotrophoblast cultures allowed the inventors to identify proteins which can constitute trisomy 21 serum markers.

The inventors indeed observed that the synthesis of certain peptide hormones, which is very high in normal cytotrophoblast cultures, at the time of formation of the syncytiotrophoblast, was very low in the cytotrophoblast cultures in accordance with the invention. These serum hormones should therefore be present in a lower quantity in the serum of women carrying a trisomy 21 fetus than in that of women carrying a normal fetus. However, hCG, which is one of the peptide hormones whose expression is reduced in the cytotrophoblast cultures in accordance with the invention, is by contrast known to be present in a higher quantity in the serum of women carrying a trisomy 21 fetus than in that of women carrying a normal fetus.

The inventors emitted the hypothesis that this apparent contradiction was explained by a higher half-life of hCG in the serum, resulting from a glycosylation abnormality in the cytotrophoblasts which are trisomic for chromosome 21, such as that described by COLE et al. (1998, abovementioned publication).

In this case, the proteins which are produced by the cytotrophoblasts in nonglycosylated form will, if they are produced in a lower quantity, also be present in a lower quantity in the serum of women carrying a trisomy 21 fetus than in that of women carrying a normal fetus, whereas by contrast the proteins produced in glycosylated form, such as hCG, will be abnormally glycosylated in the trisomy 21 cytotrophoblasts, and even if they are produced in a lower quantity, will be present in a higher quantity in the maternal blood.

To verify this hypothesis, the inventors carried out the assay, in the sera of women carrying a trisomy 21 fetus and in the sera of women carrying a normal fetus, of hCG and of another glycosylated hormone, the placental growth hormone, as well as that of the following hormones: human placental lactogen (hPL) and leptin, which are not produced by the cytotrophoblasts in glycosylated form.

They thus observed that in the sera of women carrying trisomy 21 fetuses, hPL and leptin were present in a significantly lower quantity, whereas by contrast, hCG and placental growth hormone were present in a higher quantity.

These results make it possible to propose novel methods of detection and novel trisomy 21 markers.

The subject of the present invention is a method for detecting a risk of trisomy 21 in a fetus, characterized in that it comprises the assaying, in a sample of a biological fluid obtained from the mother, of at least one protein chosen from:

the nonglycosylated proteins secreted by a cytotrophoblast culture in accordance with the invention in a lower quantity than by a normal cytotrophoblast culture;

the nonglycosylated proteins secreted by a cytotrophoblast culture in accordance with the invention in a higher quantity than by a normal cytotrophoblast culture;

the glycosylated proteins secreted by a cytotrophoblast culture in accordance with the invention, with the exception of hCG.

These proteins therefore appear to be utilizable as trisomy 21 markers in accordance with the invention.

Their assay may be carried out, according to the protein involved, either by detection and quantification of the entire protein or, if it is a protein which is degraded in the body, by detection of its degradation products (in particular peptide fragments).

Proteins “secreted by a cytotrophoblast culture in accordance with the invention in a lower quantity than by a normal cytotrophoblast culture” also comprise the proteins which are not secreted by cytotrophoblasts in accordance with the invention but are secreted by normal cytotrophoblasts.

Likewise, proteins “secreted by a cytotrophoblast culture in accordance with the invention in a higher quantity than by a normal cytotrophoblast culture” also comprise proteins which are secreted by cytotrophoblasts in accordance with the invention but which are not secreted by normal cytotrophoblasts.

The expression “biological fluid” is understood to mean in particular serum and urine. To carry out the method in accordance with the invention, said nonglycosylated protein will preferably be assayed from a serum sample.

Proteins which can be used may be identified without difficulty by persons skilled in the art, by comparing the protein secretion profiles of two cultures and/or by detecting the glycosylation or the absence of glycosylation, as indicated above.

A serum dose of one or more of said proteins different from the average dose encountered in women carrying a normal fetus indicates a risk of trisomy 21 in the fetus. If a glycosylated protein is involved, because of the higher half-life resulting from an abnormal glycosylation in trisomy 21 cytotrophoblasts, a serum dose of said protein greater than the average dose encountered in women carrying a normal fetus indicates a risk of trisomy 21 in the fetus.

With the aid of the CLONTECH Microarray device “Atlas Human 1.2”, the inventors thus observed that the cytotrophoblast cell expressed secreted proteins such as IGF₂, thymosin beta-10, Calgranulin B, macrophage colony stimulating factor, C. reactive protein precursor, EGF, FMLP-related receptor 1, small inducible cytokine A 5, IGF BP₁, hepatocyte growth factor like protein, PDGF, and some of the interleukins IL-1 to IL-18. They observed, in addition, that the cytotrophoblast cells expressed the amyloid precursor peptide (APP). They, also observed that this expression was much higher in the cytotrophoblast cultures in accordance with the invention than in the normal cytotrophoblast cultures.

The amyloid precursor peptide (APP) is a transmembrane protein (SWISS-PROT accession number: P05067), whose extracellular domain is released in soluble form after proteolytic cleavage by an α-secretase activity (release of a soluble protein comprising amino acids 18-699 of APP) or by a β-secretase activity (release of a soluble protein comprising amino acids 18-671 of APP), (for a review, cf. OCTAVE et al., Medecine/Sciences, 11, 1251-1259, 1995).

The extracellular domain of the APP protein, or of the fragments thereof, therefore appear to be utilizable, in accordance with the invention, as maternal trisomy 21 serum markers.

The inventors moreover identified hPL and leptin among the nonglycosylated proteins secreted by normal cytotrophoblast cultures and nonsecreted, or secreted in a lesser quantity, by the cytotrophoblast cultures in accordance with the invention. They also identified interleukin 13 among the glycosylated proteins secreted by the cytotrophoblast cultures in accordance with the invention. This protein is not secreted by normal cytotrophoblasts.

According to a preferred embodiment, the method in accordance with the invention comprises the assay, in a biological sample obtained from the mother, of at least one protein chosen from the glycosylated proteins secreted by a cytotrophoblast culture in accordance with the invention, and of at least one protein chosen from the nonglycosylated proteins secreted by a cytotrophoblast culture in accordance with the invention.

In the context of this embodiment, said glycosylated protein may be for example hCG, placental growth hormone, the extracellular domain of the APP protein or any other glycosylated protein secreted by cytotrophoblast cultures in accordance with the invention. Glycosylated proteins which can be used may be identified with no problem by persons skilled in the art from the protein secretion profile of a cytotrophoblast culture in accordance with the invention, by detecting the glycosylation or the absence of glycosylation, as indicated above.

The serum assay of the chosen proteins is carried out according to the customary methods, known per se to persons skilled in the art. In particular, it is possible to use immunological assay methods, with the aid of antibodies directed against the relevant protein. In the case of proteins which are known per se such as hPL, placental growth hormone, leptin, hCG or APP, appropriate antibodies are commercially available, or may be easily obtained by conventional techniques from purified preparations of these proteins. In the case of proteins identified solely from protein secretion profiles of cytotrophoblast cultures, which were not previously known and for which no specific antibody is available, appropriate antibodies may be obtained, for example, from proteins eluted from two-dimensional gels. These antibodies may then be used to purify, from cytotrophoblast cultures, larger quantities of the relevant protein.

The method of detection in accordance with the invention may be carried out as a supplement to another method of detecting trisomy 21, for example as a supplement to a method of assay involving other serum markers, or to a method of detection by ultrasound scan.

For example, to confirm or refute a diagnosis by ultrasound scan, it is possible to carry out the assay of hPL, which constitutes a highly discriminating marker from the 19th week of gestation.

The present invention will be understood more clearly with the aid of the additional description which follows, which refers to nonlimiting examples of production and characterization of cytotrophoblast cultures in accordance with the invention, and of use of peptide hormones produced by these cultures as trisomy 21 markers.

### EXAMPLE 1

### Production of Trisomy 21 Cytotrophoblast Cultures

Collection of Placental Tissues:

Placental tissue samples were collected during abortion for serious fetal abnormality, between 12 and 35 weeks of gestation, either in cases of trisomy 21 detected by karyotype, or as a control, in cases of fetal malformation without karyotype abnormalities. The samples were collected in accordance with the provisions of French law relating to the consent of the patients involved.

The samples for the two groups were paired so as to correspond to identical periods of gestation. It was verified, for samples collected in the case of trisomy 21, that the placental tissue was also trisomic for chromosome 21, and for the control samples, that the karyotype was normal.

Isolation and Culture of the Cytotrophoblasts

The placental villi were freed of membranes and vessels, rinsed and cut into thin slices in a HANK's balanced solution (HBSS) free of calcium and magnesium.

The cytotrophoblast cells were isolated by digestion with trypsin-DNAse, using the protocol described by KLIMAN et al. [Endocrinology, 118, 1567-1582, (1986)], modified as follows:

The digestion is carried out with the aid of a freshly prepared enzymatic preparation comprising 0.5% (w/v) of powdered trypsin (DIFCO), 5 IU/ml of DNAseI (SIGMA, 375 000 U, 2 600 Kunitz u/mg solid), 25 mM HEPES, 4.2 mM MgSO₄ and 1% (w/v) of penicillin/streptomycin, in HBSS.

The thinly-sliced villi are covered with this preparation; after 30 min of digestion at 37° C., the liquid is discarded and replaced by a fresh enzymatic preparation. Another incubation of 25 min is carried out, after which the liquid is discarded and again replaced by a fresh enzymatic preparation. Five successive digestions are then carried out using every time a fresh enzymatic preparation: 1 digestion of 20 min, 1 digestion of 15 min and 3 digestions of 10 min.

The liquids of the last 5 digestions are stored and combined, and then fractionated on a discontinuous Percoll gradient from 10 to 70%.

A homogeneous population of mononuclear cells is thus obtained. After centrifugation, the cellular pellet is taken up in 4 ml of DMEM medium. These 4 ml are deposited on the gradient comprising more than 90% of viable cytotrophoblastic cells [ALSAT et al., J. Clin. Encodocrinol. Metab., 73, 288-294, (1991)].

The cells are cultured in dishes 60 mm in diameter (3×10⁶ cells per dish, in 3 ml of DMEM medium, supplemented with 25 mM HEPES, 2 mM glutamine, 20% of heat-inactivated fetal calf serum, and in the presence of antibiotics (100 IU per ml of penicillin and 100 μg per ml of streptomycin). The cultures are maintained at 37° C. under a humid atmosphere: 95% of air (19% O₂, and 76% NH₂); 5% CO₂ over DMEM medium (Dulbecco's Modified Eagle Medium), in the presence of 25 mM HEPES, 2 mM glutamine, 20% heat-inactivated fetal calf serum, and under an atmosphere consisting of 95% air and 5% CO₂.

Comparison of the Properties of the Cytotrophoblasts Obtained from Trisomy 21 Placentas and of the Cytotrophoblasts Obtained from Control Placentas

The cytotrophoblasts isolated from the control placentas aggregate, and then after 24 to 48 hours, fuse and differentiate into syncytiotrophoblasts.

The cytotrophoblasts isolated from trisomy 21 placentas remain aggregated in the culture dishes. After 3 to 4 days of culture, only very few syncytiotrophoblasts are observed.

Desmoplaquine, and E-cadherin which normally disappear during the formation of syncytium, were tested for in the control cytotrophoblast cultures and in the trisomy 21 cytotrophoblast cultures, with the aid of anti-desmoplaquine or anti-E-cadherin monoclonal antibodies.

For the detection of desmoplaquine or of E-cadherin, the cells in culture were rinsed with PBS, fixed and permeabilized by immersion in methanol at −20° C. for 20 min. An anti-desmoplaquine or anti-E-cadherin monoclonal antibody, diluted 1/400 (SIGMA) is then applied to the treated cells, and the antigen-antibody complex is visualized with anti-mouse goat immunoglobulins labeled with fluorescein (SIGMA).

After 3 days of culture, desmoplaquine and E-cadherin are absent from the normal syncytiotrophoblasts, but present at the intercellular contacts in the trisomy 21 cytotrophoblast cultures.

These results clearly show that a delay and/or a defect in differentiation of the cytotrophoblast into syncytiotrophoblast exist(s) in the trisomy 21 cytotrophoblast cultures.

### EXAMPLE 2

### Characterization of the Expression and Secretion of Proteins in Trisomy 21 Cytotrophoblast Cultures

Study of Transcription

The total RNA is extracted from cytotrophoblast cultures after 24, 48 or 72 hours using the QIAGEN kit, according to the protocol indicated by the manufacturer.

mRNAs for human placental lactogen (hPL), α human chorionic gonadotrophin (αhCG), β human chorionic gonadotrophin (βhCG), human placental growth hormone (PGH) and leptin are quantified as described below, from the total RNA in solution, reversibly transcribed beforehand into cDNA.

The cDNA is amplified by RT-PCR in real time using the TaqMan® method (PE Applied Biosystems). This method is based on the use of the 5′3′ exonuclease activity of Taq polymerase in order to digest a labeled probe hybridized to a target sequence, during the extension phase of a PCR amplification. The labeling of the probe is designed such that its digestion generates a fluorescent signal. For each reaction, the C_{T} parameter (threshold cycle) is defined as the number of cycles for which the fluorescence generated by the digestion of the probe exceeds a threshold set beforehand; the higher the initial quantity of target molecule, the lower this number. The quantity of each target sequence in samples of unknown composition is thus determined by measuring C_{T} and using a calibration curve to determine the initial quantity of target sequence.

The primers used for the reverse transcription, as well as the amplification primers and the detection probes are chosen from the nucleic sequences of hPL), αhCG, βhCG, PGH and leptin which are available on databases, with the aid of the OLIGO 4.0 software (NATURAL BIOSCIENCES) and Primer express software (PERKIN-ELMER BIOSYSTEMS). Their specificity, and the absence of polymorphism was verified by searching with the aid of BLASTN [ALTSCHUL et al. Nucleic Acids Res. 25: 3389-3402, (1997)] on the dbEST and nr bases. The PCR amplifications were carried out in an ABI PRISM 7700 apparatus (PERKIN-ELMER BIOSYSTEMS).

The standard curve was established with the aid of 10-fold serial dilutions of cDNA obtained by reverse transcription from 1 μg of total RNA extracted from placenta collected during the 1st trimester of pregnancy. To correct the possible biases caused by an imprecise evaluation of the total quantity of RNA used in each reaction, as well as its quality (that is to say the absence of substantial degradation), an internal control was performed by quantification of transcripts of the PPIA gene (encoding human peptidyl prolyl isomerase A), and each sample was normalized on the basis of its PPIA content. For each sample, the quantity of target RNAs to be quantified and that of the control PPIA RNA was determined from the standard curve. Next, the quantity of target RNA was divided by the quantity of PPIA RNA in order to obtain the normalized value.

Study of Secretion

Measurement of the Hormones Produced by the Cytotrophoblasts

The concentration of hCG in the culture media was determined by an ELISA assay (VITAS system, BIOMERIEUX) using the protocol indicated by the manufacturer. The detection sensitivity of this test is 2 mU/ml.

The concentration of secreted hPL was determined with the aid of the hPL IRMA AMERLEX kit (AMERSHAM) in a 4-fold concentrated medium. The test sensitivity is 0.5 μg/ml).

hPL was also detected from cytotrophoblast cell lysates. The solubilized proteins (5 μg) were visualized after electrophoresis and transferred onto nitrocellulose using a rabbit polyclonal antibody directed against hPL and diluted 250 fold (DACO, FRANCE). The band corresponding to hPL is visualized by chemiluminescence (PIERCE SUPERSIGNAL, INTERKIM, FRANCE) after incubation with an anti-rabbit antibody coupled to peroxidase.

Leptin was measured in 4-fold concentrated medium using the RIA kit “Sensitive Human Leptin RIA Kit” (LINCO, ST LOUIS, USA). The assay sensitivity is 0.5 ng/ml.

For each assay, three independent measurements were performed.

Immunoblotting

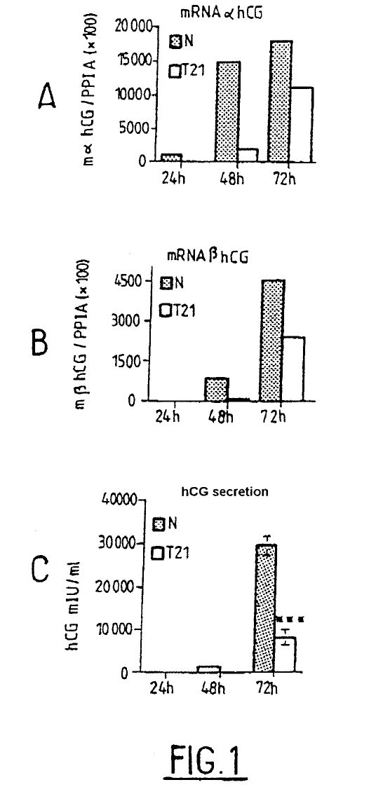

The results of these experiments are illustrated by FIGS. 1 to 3:

FIGS. 1A and 1B show the expression of the mRNAs for αhCG and βhCG during the differentiation of cytotrophoblasts isolated from control placentas (N) and from trisomy 21 placentas (T21) and cultured for 24 h, 48 h or 72 h. FIG. 1C illustrates the secretion of hCG by control cells (N) or T21 cells after 24 h, 48 h or 72 h of culture.

In the cultures of normal placental cells, the level of the transcripts of the hCG β subunit increases by more than 10³ fold between cytotrophoblast and syncytiotrophoblast differentiated in vitro. Likewise, the secretion of βhCG gradually increases in the culture medium. In the cultures of T21 placenta cells, the level of the transcripts and the secretion of hCG are reduced.

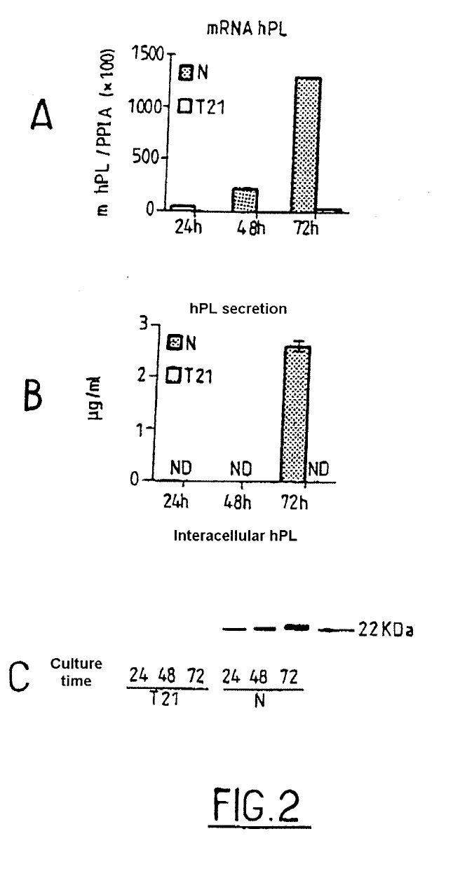

FIGS. 2A to 2C illustrate the expression of the mRNAs, the secretion in the culture medium and the intracellular production of hPL during the differentiation of cytotrophoblasts isolated from control placentas (N) or trisomic placentas (T21). FIG. 2A illustrates the level of transcription, expressed as normalized quantity of mRNA. FIG. 2B illustrates the secretion of hPL in the culture medium, expressed in μg/ml. FIG. 2C shows the detection of intracellular hPL after immunoelectrophoretic transfer.

In the T21 cells, only a very low quantity of hPL transcripts is detected after 3 days, and hPL is not detectable in the cells.

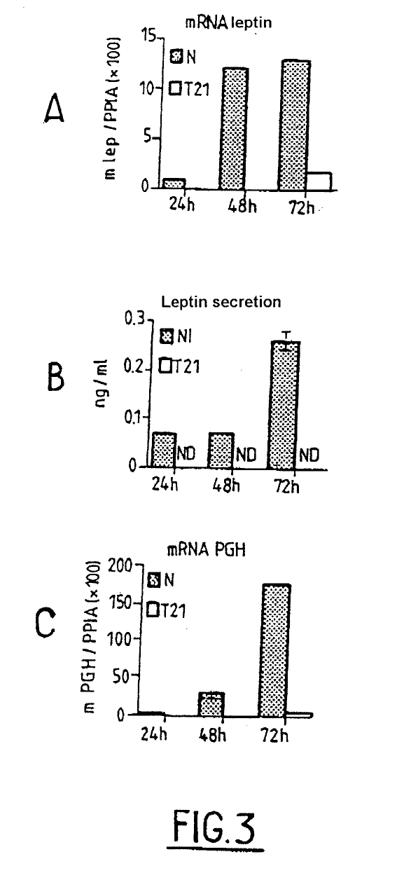

FIG. 3 illustrates the expression of the mRNAs for leptin (FIG. 3A) and for PGH (FIG. 3C), as well as the secretion of leptin (FIG. 3B) during the differentiation of cytotrophoblasts isolated from control placentas (N) or from T21 placentas cultured for 3 days.

In the cultures of normal placenta, the level of mRNA is increased by more than 20 fold (leptin) and 140 fold (placental GH) between cytotrophoblasts and syncytiotrophoblasts. In the case of trisomy 21, a defect in transcription and in the secretion of these two hormones in the culture media is observed.

These results show that, in the cells obtained from control placentas, a large increase in the mRNAs for αhCG, βhCG, hPL, leptin and PGH is observed after 72 h, which corresponds to the formation of the syncytiotrophoblast. Simultaneously, an increase in hCG, hPL and the secretion of leptin in the culture media is observed.

By contrast, in the cells obtained from trisomy 21 placentas, the defective formation of syncytiotrophoblast is associated with a large decrease in the mRNAs for αhCG, βhCG, hPL, leptin and PGH.

In parallel, a substantial decrease in the secretion of hCG is observed in the culture medium, and no secretion of hPL or of leptin in the culture medium can be detected after 3 days. Likewise, hPL is not detectable in the trisomy 21 cells, whereas it is present in the normal cells.

The secretion of PGH in the culture medium was not measured because it is known that this secretion is inhibited by glucose, which is present in this medium.

These results indicate that the defects and/or the delay in the morphological differentiation into syncytiotrophoblast of the cytotrophoblasts isolated from trisomy 21 placentas are associated with a simultaneous decrease in the expression and the secretion of the hormones specifically synthesized by the syncytiotrophoblast.

In parallel, the transcription of the genes for αhCG, βhCG, PGH, hPL and leptin was measured during placental ontogenesis, using the protocol described above, in samples of normal and T21 placentas. The results obtained indicate that throughout pregnancy, the transcription of these genes is much lower in the trisomy 21 placentas than in the normal placentas.

### EXAMPLE 3

### Use of Proteins Secreted by Trisomy 21 Cytotrophoblast Cultures as Trisomy 21 Markers

The following proteins were assayed on a sample of 86 sera collected from women carrying trisomy 21 fetuses, and 247 sera collected from women carrying normal fetuses.

Nonglycosylated Proteins

1. hPL:

The assay is carried out on serum. The sample volume is adapted as a function of the stage of pregnancy (at the beginning of pregnancy 200 μl, at the end of pregnancy 20 μl). The assay is carried out with the aid of the IRMA (immunoradiotherapy) AMERLEX-hPL-IRMA kit (Ortho Clinical Diagnostics, United Kingdom).

Each serum was assayed in duplicate with an accuracy on the inter series measurement ranging from 5.9% to 10% of coefficient of variation. The range of concentrations is from 0.5 to 16.5 μg/ml. The detection limit for the test is 0.06 μg/ml.

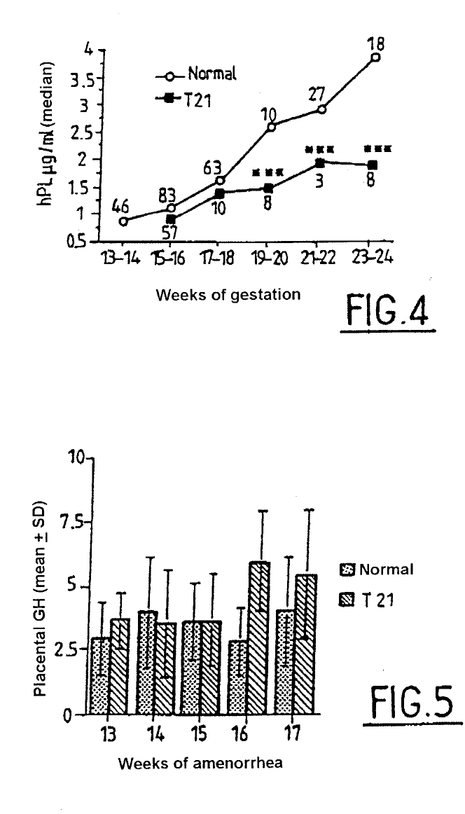

The results are illustrated by FIG. 4, which shows that in the case of T21, the hPL levels are abnormally low and that from 19-20 weeks, they become a highly discriminating marker. The maternal serum hPL levels are therefore the direct reflection of the abnormality in the formation of the syncytiotrophoblast in T21.

2. Leptin:

The assay is carried out on serum. The sample volume is 100 μl. The assay is carried out with the aid of the assay RIA Human Leptin RIA KIT (Linco, USA).

Each serum is assayed in duplicate with an accuracy on the inter series measurement ranging from 3.3% to 8.9% of coefficients of variation. The range of concentrations ranges from 0.5 to 100 ng/ml. The detection limit is 0.1 ng/ml.

A decrease in the maternal serum levels of leptin is observed in the case of fetal trisomy 21.

Glycosylated Protein

Placental GH

The assay is carried out on serum. The sample volume is 100 μl. The assay is carried out with the aid of the hPGH IRMA (BC1017) BIOCODE kit.

Each serum is assayed in duplicate with an accuracy on the inter series measurement ranging from 5.5% to 7.9% of coefficients of variation. The range of concentrations ranges from 1 to 180 ng/ml. The detection limit is 0.2 ng/ml.

The results are illustrated by FIG. 5, which confirms that the placental GH is higher in the maternal blood in the case of fetal trisomy 21 from 16 to 17 weeks.

1. A cytotrophoblast culture, characterized in that said cytotrophoblasts are trisomic for human chromosome 21, or for a region thereof comprising at least the gene encoding Cu/Zn superoxide dismutase, and in that they express, after 3 days of culture, at least one peptide hormone chosen from human placental lactogen (hPL), α human chorionic gonadotrophin (αhCG), β human chorionic gonadotrophin (βhCG), human placental growth hormone (PGH), and leptin, in a quantity less than that expressed on average by normal cytotrophoblasts derived from placentas at the same stage of gestation, and cultured under the same conditions.

2. The use of a cytotrophoblast culture as claimed in claim 1, for identifying and characterizing trisomy 21 markers.

3. A library for differential expression of nucleic acids obtained with the aid of at least one cytotrophoblast culture as claimed in claim 1.

4. A method for detecting a risk of trisomy 21 in a fetus, characterized in that it comprises the assaying, in a biological sample obtained from the mother, of at least one protein chosen from:
the nonglycosylated proteins secreted by a cytotrophoblast culture as claimed in claim 1 in a lower quantity than by a normal cytotrophoblast culture;
the nonglycosylated proteins secreted by a cytotrophoblast culture as claimed in claim 1 in a higher quantity than by a normal cytotrophoblast culture;
the glycosylated proteins secreted by a cytotrophoblast culture as claimed in claim 1, with the exception of hCG.

5. The method as claimed in claim 4, characterized in that the assay of at least one nonglycosylated protein chosen from hPL and leptin is carried out.

6. The method as claimed in claim 4, characterized in that the assay of at least one glycosylated protein from placental growth hormone, interleukin 13, and the extracellular domain of the amyloid precursor protein is carried out.

7. The method as claimed in either of claims 4 and 5, characterized in that it additionally comprises the assay of at least one protein chosen from the glycosylated proteins secreted by a cytotrophoblast culture as claimed in claim 1, and of at least one protein chosen from the nonglycosylated proteins secreted by a cytotrophoblast culture as claimed in claim 1.

8. The method as claimed in claim 7, characterized in that said glycosylated protein is chosen from hCG, placental growth hormone, interleukin 13, and the extracellular domain of the amyloid precursor protein.

9. The use of at least one protein as defined in claim 4 as marker for the in vitro diagnosis of trisomy 21.
